(19) United States
(12) Patent Application Publication
(10) Pub. No.: US 2025/0104841 A1
(43) Pub. Date: Mar. 27, 2025

(54) THERAPEUTIC CHAMBER

(71) Applicants: Cara Hewett, Winter Park, FL (US); Tracy Zboril, Winter Park, FL (US)

(72) Inventors: Cara Hewett, Winter Park, FL (US); Tracy Zboril, Winter Park, FL (US)

(21) Appl. No.: 18/809,005

(22) Filed: Aug. 19, 2024

Related U.S. Application Data
(60) Provisional application No. 63/539,698, filed on Sep. 21, 2023.

(51) Int. Cl. G16H 20/70; A61H 15/00; G16H 80/00

(52) U.S. Cl. CPC G16H 20/70; A61H 15/0078; G16H 80/00; A61H 2201/0149; A61H 2201/5012; A61H 2201/5043; A61H 2201/5097; A61H 2203/0431; A61H 2203/0456

(57) ABSTRACT

A therapeutic enclosure system includes a main body having an access panel to control an entry door. A plurality of system components are positioned within the main body including a massage chair, a display screen, speakers, lights, a camera, a microphone, an HVAC system, a red light therapy system, and a scent diffuser. Each of the system components are connected to a user controller and a system controller. The system controller communicates with an externally located computer for conducting a two way live therapy session between a user and a counselor. During the therapy session the counselor selectively controls the therapeutic system components. The system controller includes a memory for storing content for presentation to the user via the system components. During playback of the content, the system components are selectively activated automatically by the system controller. A UV disinfectant system is operable between users entering the main body.

### CROSS-REFERENCE TO RELATED APPLICATIONS

This application claims the benefit of U.S. Application Ser. No. 63/539,698 filed on Sep. 21, 2023, the contents of which are incorporated herein by reference.

### TECHNICAL FIELD

The present invention relates generally to privacy and therapy pod systems, and more particularly to a therapeutic chamber.

### BACKGROUND

The statements in this section merely provide background information related to the present disclosure and may not constitute prior art.

Many individuals suffer from stress in the workplace, school and/or other environments. In many instances, there is nowhere private for such people to recover and relax. In recent years, employers have researched how productivity levels of stressed employees compare to those of unstressed employees and the numbers are shocking. For this reason, many employers have begun to offer their employees the ability to meet with counselors and/or therapists to reduce workplace stress.

Unfortunately, many employees are not able to enjoy these benefits as they cannot leave their workplace to meet with a counselor, and doing so over their work computer is not private.

Accordingly, it would be beneficial to provide a therapeutic chamber which can allow users the opportunity to undergo an automated stress relieving session and/or meet with a live counselor in privacy so as to overcome the drawbacks noted above.

### SUMMARY OF THE INVENTION

The present invention is directed to a therapeutic enclosure system. One embodiment of the present invention can include a main body having a plurality of walls that define an interior space. A door can be positioned along one of the walls and can include a lock that is connected to an access panel. The access panel can include a touchscreen display which can selectively lock and unlock the door.

In one embodiment, a massage chair, a display screen, speakers, lights, a camera, and microphone can be positioned within the main body and can be connected to a system controller. The system controller can include functionality for communicating with an externally located computer which can be operated by a counselor. The system controller can include functionality for conducting a two-way live therapy session between a user sitting in the chair and the counselor. The system controller can also include functionality for permitting the counselor to control the operation of each of the system components during the therapy session.

In one embodiment, the system controller can also include a memory for storing content that can be accessed by the user via a user controller. During playback of the content, each of the system components may be accessed and controlled by the system controller. The main body can include an HVAC system and filter. The main body can also include a UV disinfectant system that is positioned within the inside space. The UV disinfectant system can be coupled to a motion detector and can disinfect the interior space between uses. The system can also include a red-light therapy system and a scent diffuser.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a side view of the therapeutic enclosure system that is useful for understanding the inventive concepts disclosed herein.

FIG. 2 is a side view of the therapeutic enclosure system with the door open, in accordance with one embodiment of the invention.

FIG. 3 is a side view of the interior space of the therapeutic enclosure system, in accordance with one embodiment of the invention.

FIG. 4 is another side view of the interior space of the therapeutic enclosure system, in accordance with one embodiment of the invention.

FIG. 5 is a simplified block diagram of the system controller of the therapeutic enclosure system, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “connector” includes any number of different elements that work alone or together to repeatedly join two items together in a nonpermanent manner. Several nonlimiting examples of connectors include, but are not limited to, thread-to-connect, twist-to-connect, and push-to-connect type devices, opposing strips of hook and loop material (e.g., Velcro®), attractively oriented magnetic elements or magnetic and metallic elements, buckles, clamps, clips, and compression fittings among others, for example.

As described herein, the term “content,” and derivatives thereof shall include any type of sound and/or images (e.g., still, motion, live, generated), which can be presented to a user of the therapeutic chamber via one or more of the chamber components. In various embodiments, content presented to users by the system can be designed to be therapeutic in nature so as to convey a sense of calm, serenity and safety. Content can include live or recorded therapy sessions by counselors or therapists which can be tailored to specific needs such as workplace violence recovery, mental health, disaster relief, and the like. Of course, any other types of content such as non-therapeutic workplace training and the like can also be conducted via the system components.

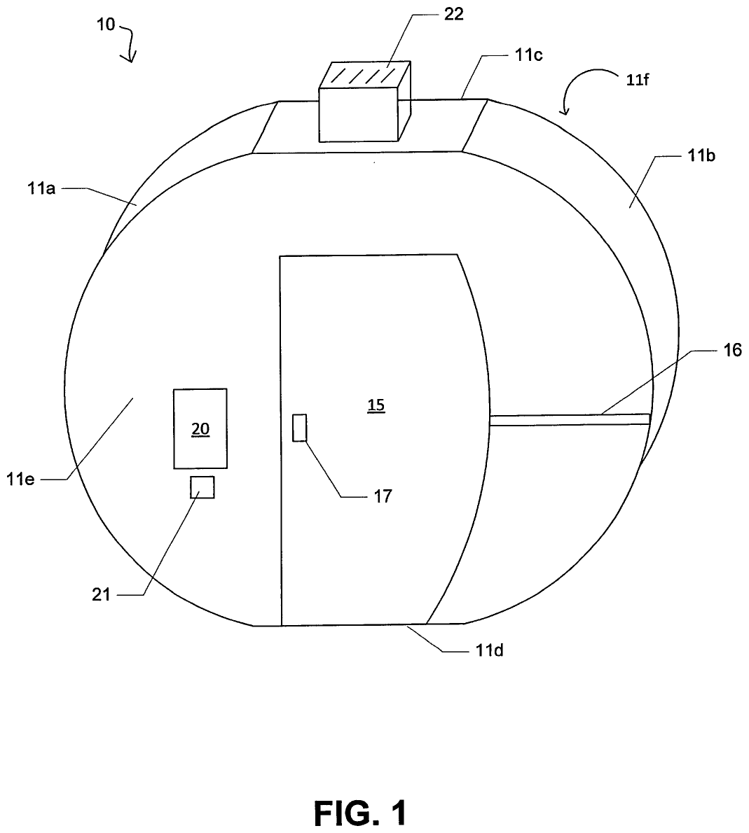

FIGS. 1-5 illustrate one embodiment of a therapeutic chamber 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

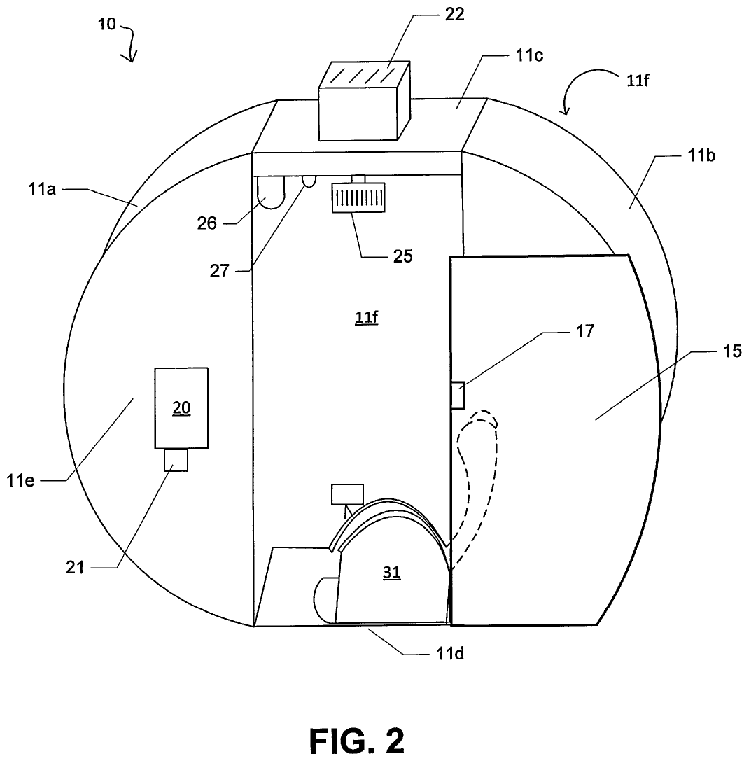

As shown at FIGS. 1 and 2, the chamber can include a main body 11 having a front wall 11a, a back wall 11b, a top wall 11c, a bottom wall 11d and a pair of side walls 11e and 11f that define an interior space. In the preferred embodiment, the main body will be constructed from lightweight materials such as plastic or composite panels that are joined together via connectors so as to enable the body to be easily shipped, assembled, disassembled and moved.

In this regard, it is preferred that each of the below described system components such as the access panel 20, POS system 21, HVAC system 22, UV system 26, motion sensor 27, camera 33, speakers 34, chamber lights 35, scent diffuser 37 and/or light therapy system 38 can be pre-installed onto the respective wall panel and electrically connected together via cable connectors during the final assembly of the chamber at the user location. Of course, other construction materials and methodologies are contemplated such that the body can be permanently installed and/or wherein one or more of the noted components are installed after the chamber has been constructed.

In either instance, the walls of the main body can preferably be lined with acoustic panels such as foam and fabric, PET or biophilic elements to ensure sounds within the chamber cannot be heard by anyone outside. These panels may be provided along the inside surface of the walls or may be provided within the walls themselves (e.g., between the inside and outside facing surfaces of the walls).

In one embodiment, door 15 can be positioned along one of the side walls and will preferably be constructed from the same material as the main body and will also include the acoustic panels. As shown, the door may be positioned along a sliding track 16 so as to slide between an open and closed position.

In one embodiment, an access panel 20 can be provided along the main body. The access panel can include a user interface preferably in the form of a touchscreen display that is connected to the below described system controller 50. The access panel can include functionality for selectively unlocking the door lock 17 to permit authorized users to enter the chamber. In one embodiment, the access panel can require a user to enter an authorization code provided by their employer, or to scan their employee badge to be allowed entry. The access panel can also notify users when the chamber is occupied.

In one embodiment, the access panel can include or can be connected to a Point-of-sale system 21 wherein users can pay for therapy sessions via cash or credit card before being granted access to the chamber. Such a feature being particularly advantageous for public venues such as malls or airports, for example, rather than a work-sponsored environment.

In one embodiment, the therapeutic chamber can include a dedicated HVAC system 22 capable of providing conditioned hot and cold air to the inside of the chamber. The HVAC system can be connected to the system controller 50 and can be controlled by a user and/or a counselor that is conducting a counseling session.

In one embodiment, an air purification system 25 can be positioned along the main body. The air purification system can preferably include an air filtration unit such as a HEPA filter that is connected to the HVAC system to ensure excellent and clean air within the chamber.

In one embodiment, the chamber can also include a UV light disinfection unit having one or more UV lights 26. The UV light disinfection unit can be controlled by the system controller and can be set to automatically clean the chamber between uses. In this regard, a motion sensor 27 can be positioned within the chamber as a redundancy safety feature to ensure the UV lights are not activated when a person is inside.

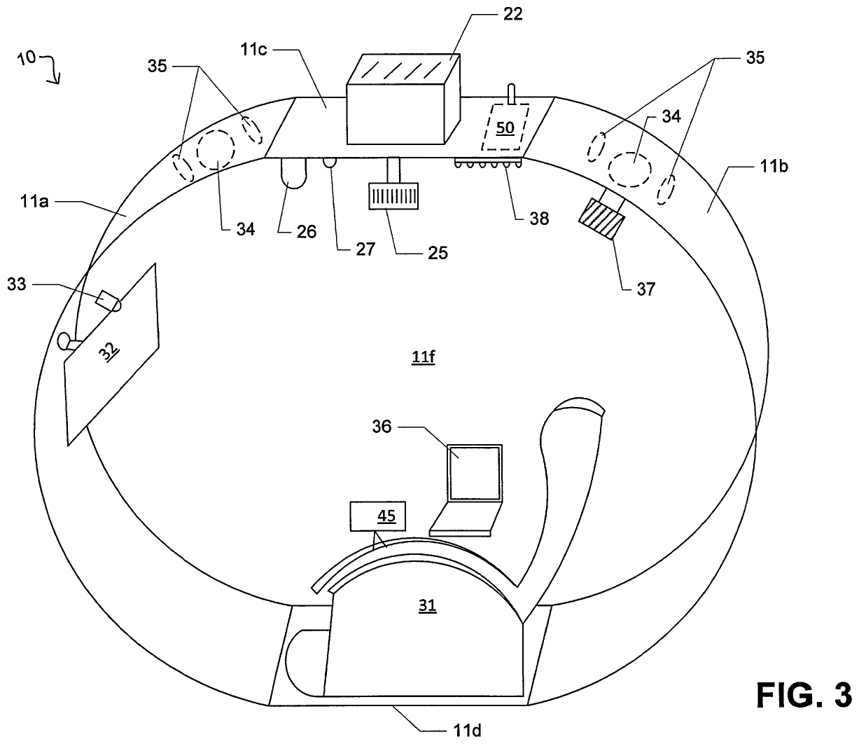

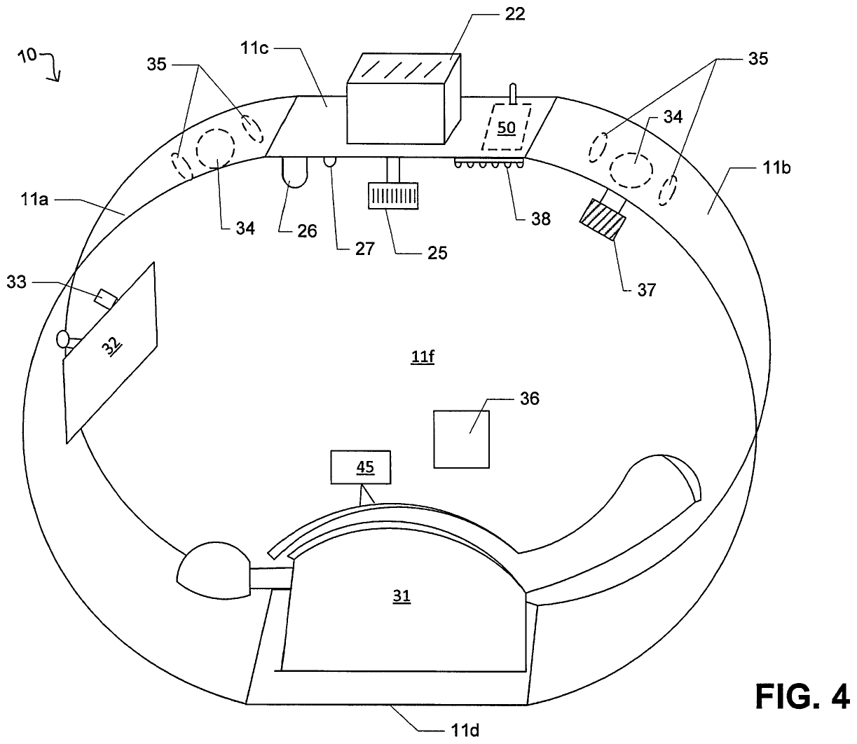

As shown best in FIGS. 3 and 4, wherein the side wall 11e is removed for ease of illustration, seat 31 can be positioned within the chamber. The seat can preferably comprise an electric massage chair having a plurality of integrated massage components such as rollers, airbags, and/or heating elements that can be used to target different areas of the body such as a user's neck, back, legs and feet, for example. The massage chair can be connected to the system controller and can transition between an upright position and a reclined and/or lay flat position.

In one embodiment, at least one display screen 32 can be provided within the chamber and connected to the controller. The display screen can function to display any type of visual content such as therapeutic scenes (e.g., beach, ocean, mountains, etc.) or informative content (e.g., live or recorded video sessions with a counselor or therapist).

In one embodiment, a video camera and microphone 33 can be provided to allow a user to conduct live two-way interactive sessions with a counselor or therapist. The camera and microphone can be connected to the controller.

In one embodiment, a plurality of speakers 34 can be provided within the chamber and connected to the controller. The speakers can function to play audible content such as music, nature sounds and/or spoken words which may be provided alone or in conjunction with any type of content.

In one embodiment, a plurality of chamber lights 35 can be provided within the chamber and connected to the controller. Each of the chamber lights can preferably include LED lights which can be connected to the controller to provide illumination within the chamber in any number of different colors and intensities. In the preferred embodiment, the chamber lights can be controlled so as to provide illumination in sync with therapeutic scenes such as yellow light when displaying a beach setting, or blue light when displaying a moonlight setting, for example.

In one embodiment, a folding table 36 can be positioned along the back wall 11f for use by the user sitting in the chair. The table can aid the user when receiving therapeutic content and can allow the user to take notes, for example.

In one embodiment, the chamber can include a scent diffuser 37 which can be connected to the controller. The scent diffuser can function to store and discharge one or more scents such as beach scent, ocean scent or mountain scent, for example, and can be activated in conjunction with the display 32, speakers 34 and lights 35 to trigger four senses by the user when experiencing a therapeutic session.

In one embodiment, a light therapy device 38 having an array of red therapy lights can be provided within the chamber and connected to the controller. The light therapy device can function to provide red light at therapeutic levels such as at 630 nm-680 nm to the user sitting in the chair. As is known, red light therapy is proven to reduce stress and promote collagen growth in the human body when exposed for between about 2-15 minutes. The light therapy can be conducted independently or concurrently with any type of content. Of course, any number of other therapeutic lighting values are contemplated.

In one embodiment, a control panel 45 can be provided. The control panel 45 can be connected to the system controller 50 and can preferably include a touchscreen display having a plurality of options for allowing a user to select from. For example, the control panel can include functionality for allowing a user to browse content from the controller memory such as visual content, audible content and/or informative content.

Additionally, the control panel can allow a user to selectively activate functionality for each of the HVAC system 22 (e.g., change the temperature), the massage chair 31 (e.g., types of massages, recline, intensity, etc.,) the display 32, the camera 33, the speakers 34 (content, volume, etc.,) the chamber lights 35 (color, brightness, etc.,), the scent diffuser 37 and/or the light therapy system 38.

Additionally, the content can be linked to each of the system components 31-35, 37 and 38 by the system controller and can be activated by the system controller during playback of the content. For example, if the user selects a beach scene from the system memory, the lights may turn crimson, the speakers may play crashing waves, the massage chair may recline and begin a relaxing massage, and the red therapy lights may activate. Of course, the user can override the automated activation and settings of any system components at any time via the user controller 45.

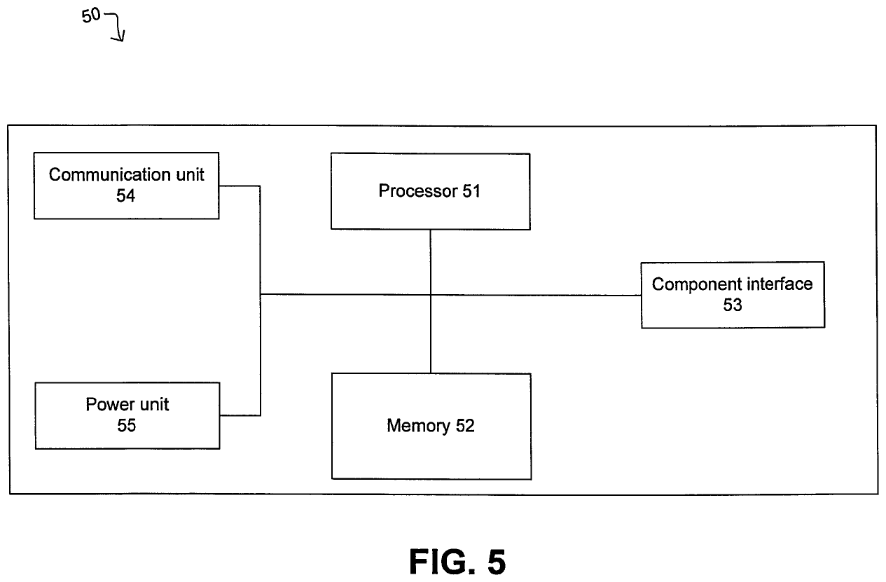

FIG. 5 is a simplistic block diagram illustrating one embodiment of the system controller 50. In one embodiment, controller 50 can include a processing unit 51 that is conventionally connected to an internal memory 52, a component interface unit 53, a communication unit 54, and a power unit 55.

Although illustrated as separate elements, those of skill in the art will recognize that one or more system components 51-55 may include, comprise, or consist of one or more printed circuit boards (PCB) containing any number of integrated circuit or circuits for completing the activities described herein. Of course, any number of other analog and/or digital components capable of performing the described functionality can be provided in place of, or in conjunction with the described elements, and the controller may be located at other locations.

The processing unit 51 can include one or more central processing units (CPU) or any other type of device, or multiple devices, capable of manipulating or processing information such as program code stored in the memory 52 in order to allow the therapeutic chamber to perform the functionality described herein.

Memory 52 can act to store operating instructions, content, and other types of information. Although illustrated in FIG. 5 as a single component, memory 52 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as an optical or solid-state hard drive, for example.

The component interface unit 53 can function to provide a communicative link between the processing unit 51 and each of chamber systems such as the access panel 20, the HVAC system 22, the UV lights 26, the motion sensor 27 the massage chair 31, the display 32, the camera 33, the speakers 34, the chamber lights 35, the scent diffuser 37, the light therapy system 38, and the control panel 45. In this regard, the component interface unit can include or can be connected to any number of different components such as one or more PIC microcontrollers, communication bus, connection cables, and the like for linking the various components.

The communication unit 54 can include any number of components capable of sending and/or receiving electronic signals with another device, either directly or over a network. In one embodiment, the communication unit can include a wireless transceiver such as a Bluetooth or Wi-Fi transceiver, for example, that allows a user to connect their smartphone or portable electronic device to the chamber during use. Such a feature can allow the user to provide or upload their own content for playback via the systems audiovisual components and/or for transmission to a counselor operating at a remote terminal.

In one embodiment, the communication unit 54 can also include a wired or wireless network adapter capable of communicating with an externally located computer over a network such as a WAN, LAN and/or the internet, for example. Such a feature allows a counselor or therapist to conduct a therapy session with a user inside the chamber.

In such an embodiment, the controller includes functionality for allowing the counselor to control each of the chamber's systems such as the display 32, the camera 33, the speakers 34, the chamber lights 35, the scent diffuser 37, and/or the light therapy system 38. Such a feature is an important feature, as the counselor can control the audiovisual environment the user experiences in conjunction with the therapy session they are providing. In this regard, by allowing the remotely located counselor to control what the user hears (e.g., speakers) sees (display and lights), smells (scent diffuser) and feels (e.g., HVAC and massage chair), the counselor is able to provide truly interactive therapy with the user.

Power regulation unit 55 can preferably include an A/C electrical power transformer and cord capable of allowing the therapeutic chamber and associated components to be powered from an electrical outlet that is connected to a power grid. Additionally, or alternatively, the power source may include a battery bank for operating the system when the commercial power is down or is not otherwise available. Such a feature being particularly advantageous when the therapeutic chamber is deployed to disaster areas where power is unstable.

As to a further description of the manner and use of the present invention, the same should be apparent from the above description. Accordingly, no further discussion relating to the manner of usage and operation will be provided.

As described herein, one or more elements of the device 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A therapeutic enclosure system, comprising:
a main body having a plurality of sides that define an interior space;
a door for permitting access to the interior space;
a seat that is positioned within the interior space;
a display screen that is positioned within the interior space;
at least one speaker that is positioned within the interior space;
a control panel that is positioned within the interior space; and
a system controller that is in communication with each of the control panel, the speaker, and the display screen.

2. The system of claim 1, wherein the system controller includes a wireless communication unit.

3. The system of claim 2, wherein the wireless communication unit is configured to send and receive information with an external computer.

4. The system of claim 3, further comprising:
a camera and microphone that are positioned within the interior space.

5. The system of claim 4, wherein the system controller includes functionality for utilizing the display, the at least one speaker, the camera and the microphone to conduct a two way therapy session between a user sitting on the chair within the interior space, and a counselor utilizing the external computer.

6. The system of claim 5, wherein the two-way therapy session comprises a live exchange of video and sound between the user and the counselor.

7. The system of claim 1, wherein the system controller includes a memory that is configured to store content.

8. The system of claim 7, wherein a user can utilize the control panel to select the stored content for playback on the display, and at least one speaker.

9. The system of claim 1, wherein the chair comprises a massage chair.

10. The system of claim 9, wherein the massage chair is configured to transition between an upright orientation and a lay flat orientation.

11. The system of claim 1, further comprising:
an access panel that is positioned along an exterior portion of the main body.

12. The system of claim 11, wherein the access panel includes functionality for selectively locking and unlocking the door.
